(19) United States
(12) Patent Application Publication
(10) Pub. No.: US 2026/0055035 A1
(43) Pub. Date: Feb. 26, 2026

(54) METHODS FOR PRODUCING 1-HEXENE

(71) Applicant: SABIC GLOBAL TECHNOLOGIES B.V., Bergen op Zoom (NL)

(72) Inventors: Zheng Liu, Sugar Land, TX (US); Michael Edward Huckman, Houston, TX (US); Andrei Merenov, Riyadh (SA); Shahid Azam, Riyadh (SA); Abdulmajeed Mohammed Al-Hamdan, Riyadh (SA)

(21) Appl. No.: 19/106,974

(22) PCT Filed: Sep. 1, 2023
(86) PCT No.: PCT/IB2023/058679

§ 371 Date: Feb. 26, 2025

Related U.S. Application Data
(60) Provisional application No. 63/403,556, filed on Sep. 2, 2022.

(51) Int. Cl. C07C 2/26; B01D 3/14; C07C 7/00; C07C 7/04

(52) U.S. Cl. CPC C07C 2/26; B01D 3/148; C07C 7/005; C07C 7/04

(57) ABSTRACT

Systems and methods of producing 1-hexene are disclosed. The methods include oligomerizing ethylene to produce 1-hexene in a reactor unit, which involves flowing a first solvent system into the reactor unit, the first solvent system adapted to improve selectivity of 1-hexene in the oligomerizing of the ethylene. The methods can include flowing part of a second solvent system into the reactor unit under washing mode, the second solvent system adapted to dissolve by-products in the reactor unit being washed. The methods can include flowing part of a second solvent system into the catalyst preparation unit, the second solvent system adapted to dissolve catalysts in the catalyst preparation step. The systems can include a reactor, a wax/polymer removal unit, and at least two C6 distillation columns in series for producing 1-hexene.

### BACKGROUND OF THE INVENTION

### A. Field of the Invention

The invention generally concerns the production of 1-hexene. More specifically, the invention involves the use of solvent systems and un-traditional polymer removal units to enhance the production of 1-hexene from the oligomerization of ethylene.

### B. Description of Related Art

Methods for the production of linear α-Olefins (LAOs) by ethylene oligomerization are known and have been described by industrial and research entities. The oligomerization of ethylene typically involves contacting the ethylene with a catalyst, co-catalyst, and solvent. Ethylene oligomerization usually results in various products. Thus, the technology developed with respect to the oligomerization process has included methods of separating and handling these various products. One of the olefins produced in the oligomerization of ethylene is 1-hexene, which is typically 10 wt. % to 27 wt. % product fraction of the total LAO products produced in the commercial process (see e.g., A. Meiswinke et al., 2011 (describing one commercial process—α-SABLIN) and can be on-purposely promoted by catalyst comprising a chromium compound, a ligand compound, and a solvent selected from the group consisting of aromatic hydrocarbons (see e.g., U.S. Pat. No. 8,778,827 B2 and U.S. Pat. No. 8,637,721 B2). Wax formation, i.e., formation of heavy, long-chain, high carbon-number products and polymer formation (polyethylene, branched and/or cross-linked PE are detected in selective ethylene trimerization, which leads to fouling of equipment (see e.g., U.S. Pat. No. 8,637,721 B2).

With respect to the process for separation and purification of 1-hexene and other by-products, there is a need in the art for improvements with respect to 1-hexene yield and purity, and effective wax/polymer removal.

### SUMMARY OF THE INVENTION

The present inventors have made a discovery that provides a solution to the improvements desired in the art with respect to 1-hexene production. In embodiments of the invention, the on-purpose 1-hexene technology utilizes two solvent systems for different purposes, which results in the 1-hexene forming a higher fraction of the oligomerization products compared to conventional processes. Embodiments of the invention also include improvements to existing processes for separating 1-hexene from the various products produced by ethylene oligomerization.

Embodiments of the present invention include a method of producing 1-hexene that comprises flowing ethylene into a reactor unit that is in reaction mode and has a catalyst disposed therein and contacting the ethylene with the catalyst. The method comprises oligomerizing the ethylene to produce 1-hexene, in the reactor unit, and flowing a first solvent system into the reactor unit, wherein the first solvent system is adapted to improve selectivity of 1-hexene in the oligomerizing of the ethylene. The method further includes flowing a reactor unit effluent from the reactor unit to downstream separation units, the reactor unit effluent comprising the 1-hexene and the first solvent system. The method further includes switching the reactor unit from reaction mode to washing mode. The method also comprises flowing a second solvent system into the reactor unit in washing mode. The second solvent system is adapted to dissolve by-products in the reactor unit, wherein the by-products comprise oligomers, polymers and waxes having 20 to 500 carbon atoms.

The present disclosure includes, without limitation, the following embodiments.

Embodiment 1: A method of producing 1-hexene by ethylene oligomerization, the method comprising: flowing a first solvent system into a reactor unit that is in reaction mode, the first solvent system adapted to improve selectivity of 1-hexene in the oligomerizing of the ethylene; flowing ethylene into the reactor unit, the reactor unit containing a catalyst and a first portion of a second solvent system, the second solvent system being different from the first solvent system; contacting the ethylene with the catalyst; oligomerizing the ethylene to produce 1-hexene, in the reactor unit; flowing a reactor unit effluent from the reactor unit, the reactor unit effluent comprising the 1-hexene and the first and second solvent systems; optionally, switching the reactor unit from reaction mode to washing mode; and optionally flowing a second portion of the second solvent system into the reactor unit in washing mode, the second solvent system adapted to dissolve by-products in the reactor unit, the by-products comprising oligomers, polymers and waxes having 20 to 500 carbon atoms.

Embodiment 2: The method of Embodiment 1, further comprising: separating unreacted ethylene from the reactor unit effluent using a first distillation column, to form a recycle ethylene stream comprising primarily ethylene and a first product stream comprising linear alpha olefins (LAOs), by-products, first solvent system, and second solvent system.

Embodiment 3: The method of Embodiment 2, wherein the first distillation column comprises a rectifying section adapted to absorb 1-hexene.

Embodiment 4: The method of Embodiment 2 or 3, further comprising: separating by-products from the first product stream to produce a wax/polymer stream comprising primarily wax and polymer and a second product stream comprising C₄ to C₈ hydrocarbons, first solvent system, and second solvent system.

Embodiment 5: The method of any one of Embodiments 2-4, further comprising: flowing the recycle ethylene stream to the reactor unit that is in reaction mode.

Embodiment 6: The method of Embodiment 4 or 5, wherein the separating of by-products from the first product stream comprises: flashing the first product stream in a first flashing vessel to produce the second product stream and a bottom flash stream comprising second solvent system and by-products; and separating some of the second solvent system from the bottom flash stream to form a solvent system recovery stream comprising first solvent system and second solvent system and a wax/polymer stream comprising wax, polymer and second solvent system in a manner that allows sufficient second solvent system to remain in the wax/polymer stream such that the wax/polymer stream is fluid.

Embodiment 7: The method of any one of Embodiments 4-6, further comprising: separating 1-butene from the second product stream to form a 1-butene stream comprising primarily 1-butene and a third product stream comprising C₆ to C₈ hydrocarbons, first solvent system, and second solvent system.

Embodiment 8: The method of Embodiment 7, further comprising: separating the third product stream by a separation unit comprising at least two C₆ distillation columns in series wherein separating of the third product stream comprises separating C₆ hydrocarbons from the third product stream, by a first C₆ distillation column, to form a fourth product stream comprising primarily C₆ hydrocarbons and a first bottom stream comprising C₇ to C₈ hydrocarbons, first solvent system, and second solvent system; and separating 1-hexene from the fourth product stream, by a second C₆ distillation column, to form a fifth product stream comprising 1-hexene and a C₆ product stream comprising other C₆ components such as n-hexane, 2-ethyl-1-butene, and one or more trans-hexene and cis-hexene isomers.

Embodiment 9: The method of Embodiment 8, wherein the fifth product stream comprises 99.5 wt. % of to 99.9 wt. % of 1-hexene.

Embodiment 10: The method of Embodiment 8 or 9, further comprising: separating first solvent system and second solvent system from the first bottom stream to form a first solvent system recycle stream comprising primarily first solvent system and a second solvent system recycle stream comprising primarily second solvent system.

Embodiment 11: The method of Embodiment 10, further comprising flowing a first portion of the first solvent system recycle stream to the reactor unit that is in reaction mode.

Embodiment 12: The method of Embodiment 10 or 11, further comprising: flowing a second portion of the first solvent system recycle stream to the rectifying section of the first distillation column for absorption of the 1-hexene.

Embodiment 13: The method of any of Embodiments 1 to 12, wherein the first solvent system comprises a paraffinic solvent system and the second solvent system comprises an aromatic solvent system.

Embodiment 14: The method of Embodiment 13, wherein the paraffinic solvent system comprises n-heptane and the aromatic solvent system comprises xylene.

Embodiment 15: The method of any of Embodiments 1 to 14, further comprising: flowing a portion of the second solvent system into a catalyst preparation unit, the second solvent system adapted to dissolve one or more catalysts prepared in the catalyst preparation unit.

Embodiment 16: A system for producing 1-hexene by ethylene oligomerization, the system comprising: a reactor unit; a source of a first solvent system adapted to improve selectivity of 1-hexene in the oligomerizing of the ethylene in fluid communication with the reactor unit; a source of ethylene in fluid communication with the reactor unit; a catalyst and a second solvent system within the reactor unit, the second solvent system being different from the first solvent system; and a reactor unit effluent in fluid communication with the reactor unit, the reactor unit effluent comprising 1-hexene and the first and second solvent systems.

Embodiment 17: The system of Embodiment 16, wherein the first solvent system and the ethylene are pre-mixed to form a feed source, the feed source in fluid communication with the reactor unit.

Embodiment 18: The system of Embodiment 16 or 17, further comprising a catalyst preparation unit in fluid communication with the reactor unit, the catalyst preparation unit comprising a mixture of the catalyst and the second solvent system.

These and other features, aspects, and advantages of the present disclosure will be apparent from a reading of the following detailed description together with the accompanying figures, which are briefly described below. The present disclosure includes any combination of two, three, four or more features or elements set forth in this disclosure, regardless of whether such features or elements are expressly combined or otherwise recited in a specific example implementation described herein. This disclosure is intended to be read holistically such that any separable features or elements of the disclosure, in any of its aspects and example implementations, should be viewed as combinable, unless the context of the disclosure clearly dictates otherwise.

This and other embodiments of the invention are discussed throughout this application. Any embodiment discussed with respect to one aspect of the invention applies to other aspects of the invention as well and vice versa. Each embodiment described herein is understood to be embodiments of the invention that are applicable to other aspects of the invention. It is contemplated that any embodiment or aspect discussed herein can be combined with other embodiments or aspects discussed herein and/or implemented with respect to any method or composition of the invention, and vice versa. Furthermore, compositions of the invention can be used to achieve methods of the invention.

The following includes definitions of various terms and phrases used throughout this specification and the claims.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %,” “vol. %,” or “mol. %” refer to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The words of the present invention can “comprise,” “consist essentially of,” or “consist of” particular ingredients, components, compositions, etc. disclosed throughout the specification.

The term “solvent system” means a single solvent or a combination of solvents. Non-limiting examples of solvent systems include n-heptane alone or n-heptane with one or more other paraffinic solvents; and xylene alone or xylene with one or more other aromatic solvents.

The term “paraffinic solvent” refers to alkane solvents, such as C₈ to C₈ cyclic or straight chain alkanes, including n-heptane, cycloheptane, isoheptane, n-hexane, methylcyclohexane, and the like.

The term “aromatic solvent” refers to solvents including at least one aryl ring, such as toluene, benzene, xylene, monochlorobenzene, dichlorobenzene, chlorotoluene, and the like.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawings.

FIG. 1 shows system for producing 1-hexene, according to embodiments of the invention; and

FIG. 2A and FIG. 2B show a method for producing 1-hexene, according to embodiments of the invention.

While the invention is susceptible to various modifications and alternative forms, specific embodiments thereof are shown by way of example in the drawings. The drawings may not be to scale.

### DETAILED DESCRIPTION OF THE INVENTION

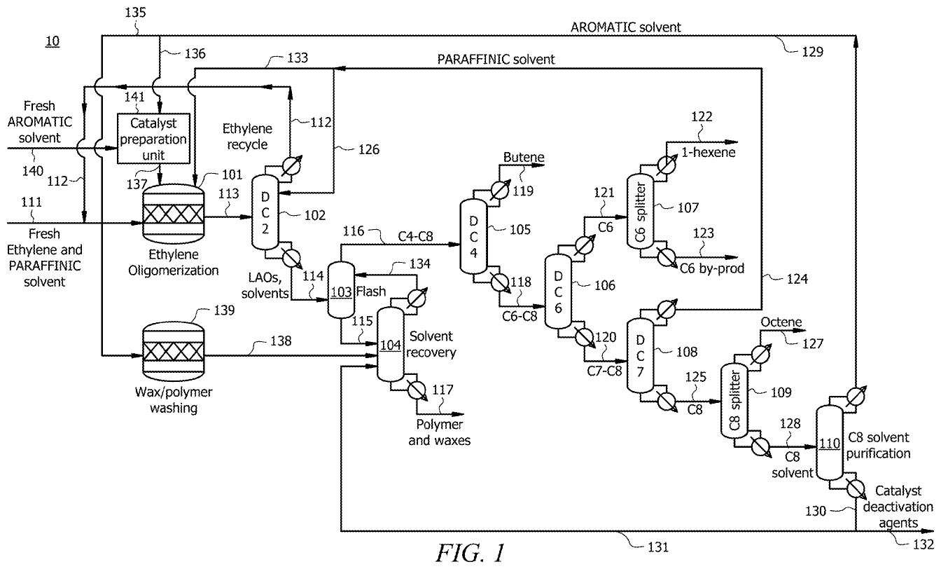

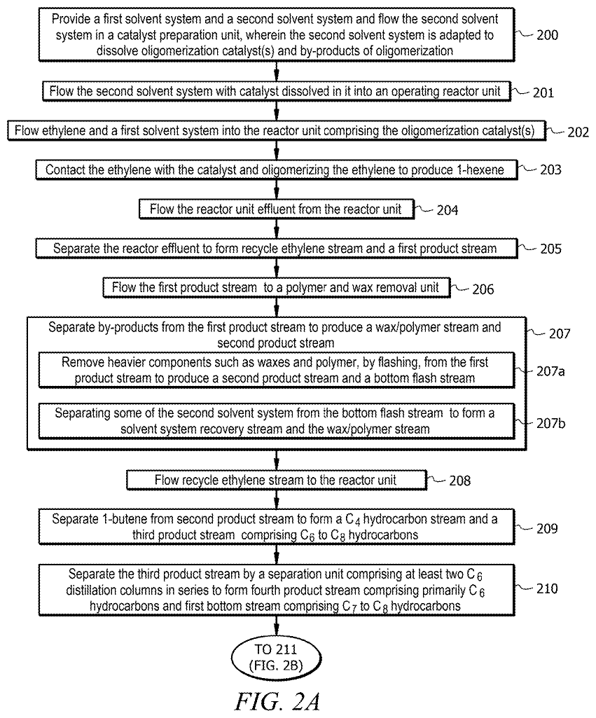

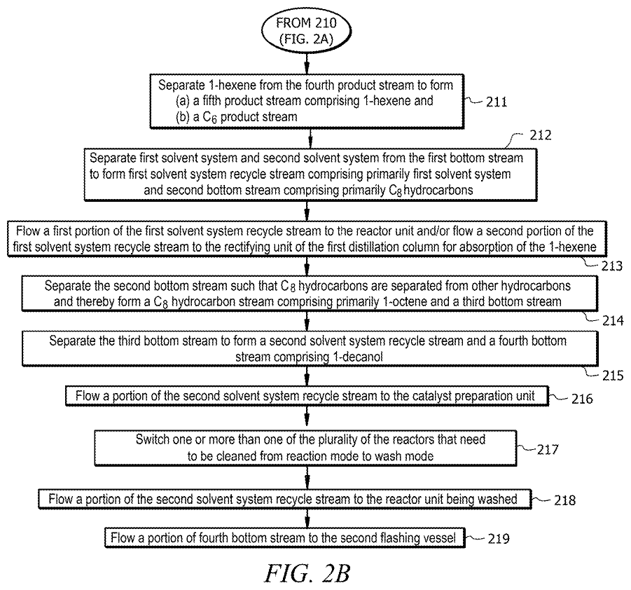

The present inventors have at least one solution to the need identified above with respect to 1-hexene production. The solution, in embodiments of the invention, includes utilizing, in a reactor unit in which the oligomerization of ethylene occurs, two solvent systems for different purposes. A first solvent system, according to embodiments of the invention, is adapted to improve selectivity of 1-hexene in the oligomerizing of the ethylene. And a second solvent system, according to embodiments of the invention, is adapted to dissolve by-products in the reactor unit, wherein the by-products comprising oligomers, polymers and waxes having 20 to 500 carbon atoms. FIG. 1 shows system 10 for producing 1-hexene, according to embodiments of the invention. FIG. 2A and FIG. 2B show a method for producing 1-hexene, according to embodiments of the invention. The method may be implemented using system 10.

These and other non-limiting aspects of the present invention are discussed in further detail in the following sections.

A. System For Producing 1-hexene

FIG. 1 shows system 10 for producing 1-hexene, according to embodiments of the invention. System 10, in embodiments of the invention, includes reactor unit 101, which is adapted to oligomerize ethylene that is present in feed 111. Feed 111, according to embodiments of the invention, comprises ethylene and a first solvent system. According to embodiments of the invention, reactor unit 101 has disposed therein an oligomerizing catalyst and/or co-catalyst in a second solvent system. In embodiments of the invention, the oligomerizing catalyst and/or co-catalyst in a second solvent system is comprised in catalyst preparation unit effluent 137, which flows from catalyst preparation unit 141. Catalyst preparation unit 141, according to embodiments of the invention, is adapted to receive fresh second solvent system stream 140 and second solvent system first recycle portion 136 (a portion of second solvent system recycle stream 129) and further adapted for the oligomerization catalyst and/or co-catalyst that is used in reactor unit 101. According to embodiments of the invention, reactor unit 101 is adapted to oligomerize ethylene in feed 111 and thereby form reactor unit effluent 113. In embodiments of the invention, system 10 comprises a plurality of reactors; for example, system 10 can comprise four individual reactors. In this way, system 10 can be operated where at least one reactor is being washed for the removal of wax and/or polymers (washing mode) while other reactors are being operated to oligomerize ethylene (reaction mode). Thus, as used herein “reaction mode” means configured to oligomerize ethylene and “washing mode” means configured to be washed so as to remove wax and/or polymer.

FIG. 1 shows reactor unit 101 as a reactor operating in reaction mode and reactor unit 139 operating in washing mode to remove wax/polymer. Reactor unit 139, according to embodiments of the invention, and as shown in FIG. 1, is in wax/polymer washing mode, and thus, is configured to receive second solvent system second recycle portion 135 (a portion of second solvent system recycle stream 129), which is used to carry out the washing. In embodiments of the invention, system 10 is configured such that any reactor unit can be switched from reaction mode to washing mode. For example, when reactor unit 101 is switched to washing mode, to remove wax/polymer therein, it would have the input and output shown in FIG. 1 for reactor unit 139, in embodiments of the invention. And when reactor unit 139 is switched to a reactor operating in reaction mode, according to embodiments of the invention, reactor unit 139 would then have the inputs and outputs shown in FIG. 1 for reactor unit 101. According to embodiments of the invention, washing effluent 138 (from the reactor being washed, in FIG. 1, reactor unit 139) is flowed to second flashing vessel 104 for processing.

According to embodiments of the invention, system 10 further comprises first distillation column 102, which is in fluid communication with reactor unit 101, adapted to receive reactor unit effluent 113 from reactor unit 101, and adapted to separate reactor unit effluent 113 into recycle ethylene stream 112 and first product stream 114. In embodiments of the invention, first distillation column 102 comprises a rectifying section above the feed entry point adapted to absorb 1-hexene. First distillation column 102, according to embodiments of the invention, comprises a stripping section below the feed entry point where ethylene is stripped from reactor unit effluent 113 and an absorption section above the feed entry point where heavier components including 1-hexene are removed from the stripped ethylene by contacting it with the first solvent system in first solvent system column recycle 126.

System 10, in embodiments of the invention, comprises a polymer and wax removal unit that comprises first flashing vessel 103 in fluid communication with first distillation column 102 such that first product stream 114 flows from first distillation column 102 to first flashing vessel 103. First flashing vessel 103, in embodiments of the invention, is adapted to remove heavier components such as waxes and polymers from first product stream 114 to produce second product stream 116 and bottom flash stream 115. First flashing vessel 103, in embodiments of the invention, is in fluid communication with second flashing vessel 104, such that bottom flash stream 115 can flow from first flashing vessel 103 to second flashing vessel 104, which is adapted to recover solvent from bottom flash stream 115 and thereby produce wax/polymer stream 117 and solvent system recovery stream 134. As shown, at least a portion of solvent system recovery stream 134 can be recycled to the first flashing vessel 103. According to embodiments of the invention, first flashing vessel 103 and second flashing vessel 104 can comprise one or more flash/distillation drums. However, it should be noted that first flashing vessel 103 and second flashing vessel 104 of the polymer and wax removal unit can be replaced by other equipment or combined with other equipment that can perform the same function.

According to embodiments of the invention, system 10 comprises second distillation column 105, which is fluid communication with first flashing vessel 103 such that second product stream 116 can flow from first flashing vessel 103 to second distillation column 105. Second distillation column 105, in embodiments of the invention, is adapted to separate second product stream 116 to form butene stream 119 (comprising primarily 1-butene) and third product stream 118 (comprising C₆ to C₈ hydrocarbons).

In embodiments of the invention, system 10 comprises first C₆ distillation column 106 in fluid communication with second distillation column 105 such that third product stream 118 can flow from second distillation column 105 to first C₆ distillation column 106. First C₆ distillation column 106, according to embodiments of the invention, is adapted to separate third product stream 118 to produce fourth product stream 121, comprising C₆ hydrocarbons, and first bottom stream 120, comprising C₇ to C₈ hydrocarbons. According to embodiments of the invention, system 10 comprises second C₆ distillation column 107 in fluid communication with first C₆ distillation column 106 such that fourth product stream 121 can flow from first C₆ distillation column 106 to second C₆ distillation column 107. Second C₆ distillation column 107, according to embodiments of the invention, is adapted to separate fourth product stream 121 to produce fifth product stream 122, comprising 1-hexene, and C₆ product stream 123, comprising other C₆ components such as n-hexane, 2-ethyl-1-butene, and one or more trans-hexene and cis-hexene isomers.

According to embodiments of the invention, system 10 comprises third distillation column 108 in fluid communication with first C₆ distillation column 106 such that first bottom stream 120 can flow from first C₆ distillation column 106 to third distillation column 108. Third distillation column 108, in embodiments of the invention, is adapted to separate first bottom stream 120 to produce first solvent system recycle stream 124 and second bottom stream 125, comprising primarily C₈ hydrocarbons. As shown, the first solvent system recycle stream 124 can be recycled for reuse as the first solvent system in the ethylene oligomerization reactors, as well as recycled for use in removing heavier components including 1-hexene from the stripped ethylene in the first distillation column 102 as noted above.

In embodiments of the invention, system 10 comprises fourth distillation column 109 in fluid communication with third distillation column 108 such that second bottom stream 125 can flow from third distillation column 108 to fourth distillation column 109. Fourth distillation column 109, in embodiments of the invention, is adapted to separate second bottom stream 125 such that C₈ hydrocarbons are separated from other hydrocarbons, especially from the second solvent system and thereby form a C₈ hydrocarbon stream 127, comprising primarily 1-octene, and a third bottom stream 128.

According to embodiments of the invention, system 10 further comprises fifth distillation column 110 in fluid communication with fourth distillation column 109 such that third bottom stream 128 can flow from fourth distillation column 109 to fifth distillation column 110. Fifth distillation column 110, in embodiments of the invention, is adapted to separate third bottom stream 128 to form second solvent system recycle stream 129 and fourth bottom stream 130 comprising 1-decanol. As shown, the second solvent recycle stream 129 can be recycled for reuse as the second solvent system used in the catalyst preparation unit 141 and for reuse as a washing solvent in the oligomerization reactors.

B. Method For Producing 1-hexene

FIG. 2A and FIG. 2B show a method for producing 1-hexene, according to embodiments of the invention. The method, in embodiments of the invention, includes, at block 200, providing a first solvent system and a second solvent system and flowing the second solvent system into catalyst preparation unit 141. The second solvent system, according to embodiments of the invention, is adapted to dissolve the oligomerization catalysts and to dissolve oligomers, polymers, and waxes having 20 to 500 carbon atoms that are formed during ethylene oligomerization. The second solvent system, according to embodiments of the invention, comprises a C₈ aromatic solvent such as xylene. As shown in FIG. 1, the second solvent system may be fed to catalyst preparation unit 141 as fresh second solvent system stream 140 and/or a separate stream such as second solvent system first recycle portion 136 (a portion of second solvent system recycle stream 129) in embodiments of the invention. The catalyst is prepared in catalyst preparation unit 141 by dissolving the catalyst in second solvent system.

At block 201, according to embodiments of the invention, the method involves flowing catalyst preparation unit effluent 137 (which comprises second solvent system and dissolved catalyst) into reactor unit 101 (which is in reaction mode).

At block 202, the method includes, according to embodiment of the invention, flowing ethylene into reactor unit 101, which comprises the catalyst(s) for the oligomerization of ethylene. In embodiments of the invention, feed 111 comprises ethylene and first solvent system; and feed 111 is flowed into reactor unit 101 as at least one source of the ethylene. Alternatively or additionally the first solvent system can be flowed to reactor unit 101 in a different stream, for example, recycle ethylene stream 112.

In embodiments of the invention the first solvent system is adapted to improve selectivity of 1-hexene in the oligomerizing of the ethylene. According to embodiments of the invention, the first solvent system comprises paraffinic solvent, e.g., C₇ paraffinic solvent such as n-heptane.

According to embodiments of the invention, at block 203, the method involves contacting the ethylene with the catalyst and oligomerizing the ethylene to produce 1-hexene, in reactor unit 101. In embodiments of the invention, the catalyst comprises but is not limited to chromium salt, ligand, aluminum alkyl and ammonium salt.

Example catalysts useful in the present disclosure are set forth, for example, in WO2020/100010 to Al-Nezari et al., which is incorporated by reference herein and which describes ligands having an NPN(CH₃)PN backbone. Example chromium compounds include organometallic Cr(III) species, such as Cr(III)acetylacetonate, Cr(III)octanoate, CrCl₃(tetrahydrofuran)₃, Cr(III)-2-ethylhexanoate, Cr(III)chloride, or any combination thereof. The catalyst can include an activator (also known in the art as a co-catalyst), such as an aluminum compound. Non-limiting examples of aluminum compounds include trimethylaluminum, triethylaluminum, triisopropylaluminum, triisobutylaluminum, diethyl aluminum chloride, ethylaluminum sesquichloride, ethylaluminum dichloride, methylaluinoxane, or a mixture thereof. In some embodiments, the activator can be a modified methylaluminoxane, such as MMAO-3 A (CAS No. 146905-79-5).

According to embodiments of the invention, the method involves, at block 204, flowing reactor unit effluent 113 from reactor unit 101, wherein reactor unit effluent 113 comprises the 1-hexene, first solvent system, and second solvent system. At block 205, in embodiments of the invention, the method involves flowing reactor unit effluent 113 to first distillation column 102, which is adapted to receive and separate reactor unit effluent 113. And in embodiments of the invention, first distillation column 102 separates reactor unit effluent 113 to form recycle ethylene stream 112 comprising primarily ethylene (unreacted ethylene) and first product stream 114 comprising linear alpha olefins (LAOs), by-products, first solvent system, and second solvent system. In embodiments of the invention, first distillation column 102 comprises a rectifying section that absorbs 1-hexene, at block 205. According to embodiments of the invention, at block 205, first distillation column 102 comprises a stripping section below the feed entry point and is used to strip ethylene from reactor unit effluent 113 and an absorption or rectifying section above the feed entry point is used to remove heavier components from the stripped ethylene by contacting it with the first solvent system in first solvent system column recycle 126. In this way, in embodiments of the invention, recycle ethylene stream 112 comprises 97 to 99 wt. % ethylene (purified ethylene), which can be compressed and returned to reactor unit 101. A small purge stream of the recycle ethylene stream 112 (not shown) is sent to a flare system in order to prevent accumulation of lighter components such as nitrogen in the process. First product stream 114 flowing from the bottom of first distillation column 102, according to embodiments of the invention, comprises very little ethylene, e.g., 1 to 3 wt. % ethylene.

In embodiments of the invention, the method involves, at block 206, flowing first product stream 114 to a polymer and wax removal unit and, at block 207, separating, in the polymer and wax removal unit, by-products from first product stream 114 to produce (1) wax/polymer stream 117 comprising primarily wax and polymer and (2) second product stream 116 comprising C₄ to C₈ hydrocarbons, first solvent system, and second solvent system. In this way, the polymer and wax removal unit protects the main separation train of the process from potential fouling due to the presence of the polymer and waxes.

According to embodiments of the invention, the polymer and wax removal unit comprises first flashing vessel 103 and second flashing vessel 104, where first flashing vessel 103 receives first product stream 114 from first distillation column 102. First flashing vessel 103, in embodiments of the invention, at block 207a, removes heavier components such as waxes and polymer from first product stream 114 by flashing to produce (1) second product stream 116 and (2) bottom flash stream 115 comprising second solvent system and by-products. In embodiments of the invention second product stream 116 comprises 0 to 0.1 wt. % polymers (i.e., only traces) and bottom flash stream 115 comprises 5 to 10 wt. % polymer. In this way, embodiments of the invention can avoid excessive concentration of the polymer (above 10 wt. % or temperatures below 140° C.) in bottom flash stream 115. Excessive concentration of polymer can lead to the precipitation of the polymer from the solution and can thereby cause excessive fouling of equipment. On the other hand, excessive entrainment of polymer in second product stream 116 can lead to fouling problems in the main separation train. At block 207b, in embodiments of the invention, second flashing vessel 104 receives bottom flash stream 115 from first flashing vessel 103 and second flashing vessel 104 recovers solvent from bottom flash stream 115 by separating some of the second solvent system from bottom flash stream 115 to form (1) solvent system recovery stream 134 comprising first solvent system and/or second solvent system and (2) wax/polymer stream 117 comprising wax, polymer and second solvent system in a manner that allows sufficient second solvent system to remain in the wax/polymer stream such that the wax/polymer stream 117 is fluid. The fluidity of wax/polymer stream 117 can reduce the risk of inadvertent equipment plugging and/or unexpected accumulation of solids in pipes and valves. In other words, removing polymer/wax as a flowable liquid can provide advantages in terms of ease of operability and/or reduced maintenance issues. According to embodiments of the invention, block 207, including block 207a and block 207b can be carried out by equipment other than or in combination with first flashing vessel 103 and second flashing vessel 104.

In embodiments of the invention, the method includes, at block 208, flowing recycle ethylene stream 112 to reactor unit 101. At block 209, in embodiments of the invention, the method involves second distillation column 105 receiving second product stream 116 from first flashing vessel 103 and second distillation column 105 separating 1-butene from second product stream 116 to form (1) a C₄ hydrocarbon stream, butene stream 119, comprising primarily 1-butene and (2) third product stream 118 comprising C₆ to C₈ hydrocarbons, first solvent system, and second solvent system. In embodiments of the invention, second product stream 116 can be cooled and condensed before entering the separation train at second distillation column 105.

In embodiments of the invention, the method involves separating third product stream 118 by a separation unit comprising at least two C₆ distillation columns in series. By using two columns in series, a higher product purity of 1-hexene can be achieved (e.g., when required for use as a PE copolymer, which needs high purity and n-hexane can be troublesome in the polymerization process). According to embodiments of the invention, separating third product stream 118, involves, at block 210, first C₆ distillation column 106 receiving third product stream 118 from second distillation column 105 and first C₆ distillation column 106 separating C₆ hydrocarbons from third product stream 118, to form (1) fourth product stream 121 comprising primarily C₆ hydrocarbons and (2) first bottom stream 120 comprising C₇ to C₈ hydrocarbons, first solvent system, and second solvent system.

According to embodiments of the invention, the method involves, at block 211, second C₆ distillation column 107 receiving, fourth product stream 121 from first C₆ distillation column 106 and second C₆ distillation column 107 separating 1-hexene from fourth product stream 121 to form (1) fifth product stream 122 comprising 1-hexene and (2) C₆ product stream 123 comprising n-hexane, 2-ethyl-1-butene, and one or more trans-hexene and cis-hexene isomers. In embodiments of the invention, fifth product stream 122 comprises 99.5 wt. % to 99.9 wt. % of 1-hexene.

According to embodiments of the invention, the method involves separating first solvent system and second solvent system from first bottom stream 120 to form first solvent system recycle stream 124 comprising primarily first solvent system. Block 212, according to embodiments of the invention, comprises third distillation column 108 receiving first bottom stream 120 from first C₆ distillation column 106 and third distillation column 108 separating first solvent system to produce (1) first solvent system recycle stream 124 and (2) second bottom stream 125, comprising primarily C₈ hydrocarbons. At block 213, in embodiments of the invention, the method involves flowing a first portion of first solvent system recycle stream 124 (first solvent system reactor recycle 133) to reactor unit 101 and/or flowing a second portion of the first solvent system recycle stream 124 (first solvent system column recycle 126) to the rectifying section of first distillation column 102 for absorption of the 1-hexene. Recycling a portion of first solvent system recycle stream 124 (first solvent system reactor recycle 133) to reactor unit 101 can help to prevent undesired chemical reactions. And recycling a second portion of the first solvent system recycle stream 124 (first solvent system column recycle 126) to the top of first distillation column 102 as an extraction medium can help to purify the ethylene distillate stream (recycle ethylene stream 112) so that the C₄+ olefins can be pushed more to the bottom of the column.

According to embodiments of the invention, at block 214, the method involves fourth distillation column 109 receiving second bottom stream 125 from third distillation column 108 and third distillation column 108 separating second bottom stream 125 such that C₈ hydrocarbons are separated from other hydrocarbons, especially from the second solvent system and thereby form (1) C₈ hydrocarbon stream 127 comprising primarily 1-octene and (2) third bottom stream 128.

At block 215, in embodiments of the invention, the method involves fifth distillation column 110 receiving third bottom stream 128 from fourth distillation column 109 and fifth distillation column 110 separating third bottom stream 128 to form (1) second solvent system recycle stream 129 and (2) fourth bottom stream 130 comprising 1-decanol. In this way, fifth distillation column 110, according to embodiments of the invention, reduces the content of catalyst deactivation agent-1-decanol. According to embodiments of the invention, at block 216, second solvent system recycle stream 129 is split and a first part of second solvent system recycle stream 129, second solvent system first recycle portion 136, flows to catalyst preparation unit 141 for dissolving the catalysts in the catalyst preparation step. In embodiments of the invention, at block 217, one or more than one of the plurality of reactors in reactor unit 101 that need to be cleaned are switched from reaction mode to washing mode (i.e., it will be configured like reactor unit 139 as shown in FIG. 1). At block 218, in embodiments of the invention, a second portion of second solvent system recycle stream 129 (second solvent system second recycle portion 135) is flowed to the reactor in washing mode, reactor unit 139, as shown in FIG. 1. FIG. 1 depicts a reaction unit in two modes; first, it depicts reactor unit 109 in reaction mode and second it depicts reaction unit 139 in wash mode. In other words, when the reactor unit is under reaction mode, it is reactor unit 101 and when the reactor unit is under wash mode, it is reactor unit 139. The washing of reactor unit 139 by second solvent system recycle stream 129 results in washing effluent 138 flowing from reactor unit 139. In embodiments of the invention, the method includes, at block 219, flowing washing effluent 138 (comprising second solvent system with wax/polymer dissolved therein) into second flashing vessel 104 for processing. According to embodiments of the invention, fourth bottom stream 130 has an elevated amount of 1-decanol and block 220 includes flowing a portion of fourth bottom stream 130 (recycle portion fourth bottom stream 131) to second flashing vessel 104 for recovery of more second solvent system and the rest of fourth bottom stream 130 can be purged out of the process in purge stream 132.

In sum, embodiments of the process disclosed herein can achieve one or more of the following: (1) separate and purify the main product 1-hexene from the reactor unit effluent, (2) separate and purify non-reacted ethylene to recycle back to the reactor, (3) separate and purify process first solvent system (paraffinic solvent) to recycle back to the reactor and the ethylene separation column (first distillation column 102), (4) separate and purify second solvent system (aromatic solvent) and recycle it back to the reactor wash system and to the catalyst preparation section and (5) separate by-products of the reaction—polymer, butene and octene.

### Example

The present invention will be described in greater detail by way of a specific example. The following example is offered for illustrative purposes only, and is not intended to limit the invention in any manner. Those of skill in the art will readily recognize a variety of noncritical parameters which can be changed or modified to yield essentially the same results.

### Example (Simulation)

The process described herein was applied to a simulation case where ethylene has been catalytically converted to 1-hexene at 92 wt. % selectivity in a system as illustrated in FIG. 1. In the ethylene oligomerization in reactor unit 101, the temperature is about 70° C. and the pressure is about 30 bar. N-heptane was chosen as the first solvent system (paraffinic) solvent for boosting the selectivity of 1-hexene, and xylene was chosen to serve as the second solvent system (aromatic) solvent for catalyst dissolving and reactor cleaning. About 2.6% of the recycled N-heptane is added to the top of first distillation column 102 to absorb the product 1-hexene, preventing it from leaving in the overhead and being destroyed in the reactor.

A dual C₆ tower design is arranged for getting a high purity 1-hexene product in fifth product stream 122, where first C₆ distillation column 106 has about 36 theoretical stages, and second C₆ distillation column 107 has about 86 theoretical stages. As the results, the fifth product stream 122 reaches a purity of 99.5 wt. % 1-hexene and the n-hexane concentration is in trace amount.

The heavy ends removal section (polymer and wax removal unit) removes almost all the polymers and waxes, where the xylene solvent loss is about 40%. However, no vacuum flash is required in this design and the polymer mixture is always flowable in liquid phase—at 9.8 wt. % of polymer in the total polymer product (wax/polymer stream 117). A regular first flashing vessel 103 running at 3 bar and second flashing vessel 104 with 12 theoretical stages running at 1.2 bar are the only two units employed. This design can provide considerable savings on CAPEX and OPEX comparing to the lesser xylene loss design that requires buying and operating a vacuum system, and represents significant advantages in terms of easier operability and reduced maintenance issues.

Flow rates and concentration of key streams are listed in Table 1 for this example.

TABLE 1

| | Stream # in FIG. 1 | | | | | | | | |
|---|---|---|---|---|---|---|---|---|---|
| | 113 | 112 | 126 | 133 | 129 | 117 | 119 | 122 | 127 |
| Mass Flows, | 52645 | 6067 | 1000 | 37726 | 5000 | 3200 | 200 | 5671 | 133 |
| kg/hr | | | | | | | | | |
| Mass | | | | | | | | | |
| Fractions | | | | | | | | | |
| ETHYLENE | 0.113 | 0.962 | 0.000 | 0.000 | 0.000 | trace | trace | 0.000 | 0.000 |
| ETHANE | 0.000 | 0.000 | 0.000 | 0.000 | 0.000 | 0.000 | 0.000 | 0.000 | 0.000 |
| 1-BUTENE | 0.004 | 0.003 | trace | 0.000 | 0.000 | trace | 0.954 | 339.242 PPM | 0.000 |
| 1-HEXENE | 0.108 | 0.000 | trace | trace | 0.000 | trace | 0.028 | 0.995 | trace |
| Other C6 | 0.001 | trace | trace | trace | 0.001 | trace | trace | 0.001 | trace |
| N-HEPTAN | 0.721 | 0.035 | 0.999 | 0.999 | trace | 0.010 | 0.017 | trace | 0.029 |
| 1-OCTENE | trace | trace | trace | trace | 0.006 | 0.004 | trace | trace | 0.236 |
| XYLENE | 0.045 | trace | trace | trace | 0.962 | 0.851 | trace | trace | 0.685 |
| C₁₂= | trace | 0.000 | trace | trace | trace | 0.006 | trace | 0.000 | trace |
| Polymer | 0.003 | 0.000 | trace | trace | 0.027 | 0.099 | trace | 0.000 | trace |
| and waxes | | | | | | | | | |
| 1-Decanol | 0.001 | 0.000 | trace | trace | trace | 0.019 | trace | 0.000 | trace |

In the context of the present invention, at least the following 15 embodiments are described. Embodiment 1 is a method of producing 1-hexene. The method includes flowing ethylene into a reactor unit that is in reaction mode and has a catalyst disposed therein. The method further includes contacting the ethylene with the catalyst and oligomerizing the ethylene to produce 1-hexene, in the reactor unit. The method still further includes flowing a first solvent system into the reactor unit, the first solvent system adapted to improve selectivity of 1-hexene in the oligomerizing of the ethylene. The method yet further includes flowing a reactor unit effluent from the reactor unit, the reactor unit effluent comprising the 1-hexene and first solvent system. The method also includes switching the reactor unit from reaction mode to washing mode and flowing a first portion of a second solvent system into the reactor unit in washing mode, the second solvent system adapted to dissolve by-products in the reactor unit, the by-products including oligomers, polymers and waxes having 20 to 500 carbon atoms. Embodiment 2 is the method of embodiment 1, further including separating unreacted ethylene from the reactor unit effluent using a first distillation column, to form a recycle ethylene stream comprising primarily ethylene and a first product stream comprising linear alpha olefins (LAOs), by-products, first solvent system, and second solvent system. Embodiment 3 is the method of embodiment 2, wherein the first distillation column includes a rectifying section adapted to absorb 1-hexene. Embodiment 4 is the method of embodiment 3, further including separating by-products from the first product stream to produce a wax/polymer stream comprising primarily wax and polymer and a second product stream comprising C₄ to C₈ hydrocarbons, first solvent system, and second solvent system. Embodiment 5 is the method of embodiment 4, further including flowing the recycle ethylene stream to the reactor unit. Embodiment 6 is the method of embodiment 5, wherein the separating of by-products from the first product stream includes flashing the first product stream in a first flashing vessel to produce the second product stream and a bottom flash stream comprising second solvent system and by-products. The method further includes separating some of the second solvent system from the bottom flash stream to form a solvent system recovery stream comprising first solvent system and second solvent system and a wax/polymer stream comprising wax, polymer and second solvent system in a manner that allows sufficient second solvent system to remain in the wax/polymer stream such that the wax/polymer stream is fluid. Embodiment 7 is the method of embodiment 5, further including separating 1-butene from the second product stream to form a 1-butene stream comprising primarily 1-butene and a third product stream comprising C₆ to C₈ hydrocarbons, first solvent system, and second solvent system. Embodiment 8 is the method of embodiment 7, further including separating the third product stream by a separation unit including at least two C₆ distillation columns in series, wherein separating of the third product stream includes separating C₆ hydrocarbons from the third product stream, by a first C₆ distillation column, to form a fourth product stream comprising primarily C₆ hydrocarbons and a first bottom stream comprising C₇ to C₈ hydrocarbons, first solvent system, and second solvent system. The method further includes separating 1-hexene from the fourth product stream, by a second C₆ distillation column, to form a fifth product stream comprising 1-hexene and a C₆ product stream comprising n-hexane, 2-ethyl-1-butene, and one or more trans-hexene and cis-hexene isomers. Embodiment 9 is the method of embodiment 8, wherein the fifth product stream comprises 99.5 wt. % of to 99.9 wt. % of 1-hexene. Embodiment 10 is the method of embodiment 8, further including separating first solvent system and second solvent system from the first bottom stream to form a first solvent system recycle stream comprising primarily first solvent system and a second solvent system recycle stream comprising primarily second solvent system. Embodiment 11 is the method of embodiment 10, further including flowing a first portion of the first solvent system recycle stream to the reactor unit. Embodiment 12 is the method of embodiment 11, further including flowing a second portion of the first solvent system recycle stream to the rectifying section of the first distillation column for absorption of the 1-hexene. Embodiment 13 is the method of any of embodiments 1 to 12, wherein the first solvent system includes a paraffinic solvent system and the second solvent system includes an aromatic solvent system. Embodiment 14 is the method of embodiment 13, wherein the paraffinic solvent system comprises n-heptane and the aromatic solvent system comprises xylene. Embodiment 15 is the method of any of embodiments 1 to 14, further including flowing a second portion of the second solvent system into a catalyst preparation unit, the second solvent system adapted to dissolve one or more catalysts prepared in the catalyst preparation unit.

All embodiments described above and herein can be combined in any manner unless expressly excluded.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein can be utilized. Accordingly, the appended claims are intended to include within their scope such processes, machines, manufacture, compositions of matter, means, methods, or steps.

1. A method of producing 1-hexene by ethylene oligomerization, the method comprising:
flowing a first solvent system into a reactor unit that is in reaction mode, the first solvent system adapted to improve selectivity of 1-hexene in the oligomerizing of the ethylene;
flowing ethylene into the reactor unit, the reactor unit containing a catalyst and a first portion of a second solvent system, the second solvent system being different from the first solvent system;
contacting the ethylene with the catalyst;
oligomerizing the ethylene to produce 1-hexene, in the reactor unit;
flowing a reactor unit effluent from the reactor unit, the reactor unit effluent comprising the 1-hexene and the first and second solvent systems;
optionally, switching the reactor unit from reaction mode to washing mode; and
optionally flowing a second portion of the second solvent system into the reactor unit in washing mode, the second solvent system adapted to dissolve by-products in the reactor unit, the by-products comprising oligomers, polymers and waxes having 20 to 500 carbon atoms.

2. The method of claim 1, further comprising:
separating unreacted ethylene from the reactor unit effluent using a first distillation column, to form a recycle ethylene stream comprising primarily ethylene and a first product stream comprising linear alpha olefins (LAOs), by-products, first solvent system, and second solvent system.

3. The method of claim 2, wherein the first distillation column comprises a rectifying section adapted to absorb 1-hexene.

4. The method of claim 3, further comprising:
separating by-products from the first product stream to produce a wax/polymer stream comprising primarily wax and polymer and a second product stream comprising C₄ to C₈ hydrocarbons, first solvent system, and second solvent system.

5. The method of claim 4, further comprising:
flowing the recycle ethylene stream to the reactor unit that is in reaction mode.

6. The method of claim 5, wherein the separating of by-products from the first product stream comprises:
flashing the first product stream in a first flashing vessel to produce the second product stream and a bottom flash stream comprising second solvent system and by-products; and
separating some of the second solvent system from the bottom flash stream to form a solvent system recovery stream comprising first solvent system and second solvent system and a wax/polymer stream comprising wax, polymer and second solvent system in a manner that allows sufficient second solvent system to remain in the wax/polymer stream such that the wax/polymer stream is fluid.

7. The method of claim 5, further comprising:
separating 1-butene from the second product stream to form a 1-butene stream comprising primarily 1-butene and a third product stream comprising C₆ to C₈ hydrocarbons, first solvent system, and second solvent system.

8. The method of claim 7, further comprising:
separating the third product stream by a separation unit comprising at least two C₆ distillation columns in series,
wherein separating of the third product stream comprises separating C₆ hydrocarbons from the third product stream, by a first C₆ distillation column, to form a fourth product stream comprising primarily C₆ hydrocarbons and a first bottom stream comprising C₇ to C₈ hydrocarbons, first solvent system, and second solvent system; and
separating 1-hexene from the fourth product stream, by a second C₆ distillation column, to form a fifth product stream comprising 1-hexene and a C₆ product stream comprising other C₆ components such as n-hexane, 2-ethyl-1-butene, and one or more trans-hexene and cis-hexene isomers.

9. The method of claim 8, wherein the fifth product stream comprises 99.5 wt. % of to 99.9 wt. % of 1-hexene.

10. The method of claim 8, further comprising:
separating first solvent system and second solvent system from the first bottom stream to form a first solvent system recycle stream comprising primarily first solvent system and a second solvent system recycle stream comprising primarily second solvent system.

11. The method of claim 10, further comprising flowing a first portion of the first solvent system recycle stream to the reactor unit that is in reaction mode.

12. The method of claim 11, further comprising:
flowing a second portion of the first solvent system recycle stream to the rectifying section of the first distillation column for absorption of the 1-hexene.

13. The method of claim 1, wherein the first solvent system comprises a paraffinic solvent system and the second solvent system comprises an aromatic solvent system.

14. The method of claim 13, wherein the paraffinic solvent system comprises n-heptane and the aromatic solvent system comprises xylene.

15. The method of claim 1, further comprising:
flowing a portion of the second solvent system into a catalyst preparation unit, the second solvent system adapted to dissolve one or more catalysts prepared in the catalyst preparation unit.
